(19) United States
(12) Patent Application Publication
(10) Pub. No.: US 2008/0033241 A1
(43) Pub. Date: Feb. 7, 2008

(54) LEFT ATRIAL APPENDAGE CLOSURE

(76) Inventors: Ruey-Feng Peh, Mountain View, CA (US); Vahid Saadat, Saratoga, CA (US)

Correspondence Address: LEVINE BAGADE HAN LLP, 2483 EAST BAYSHORE ROAD, SUITE 100, PALO ALTO, CA, 94303, US

(21) Appl. No.: 11/828,281

(22) Filed: Jul. 25, 2007

Related U.S. Application Data
(60) Provisional application No. 60/821,113, filed on Aug. 1, 2006.

(51) Int. Cl. A61B 1/00; A61B 17/128

(52) U.S. Cl. 600/109; 128/898; 606/142; 606/157; 606/41

(57) ABSTRACT

Methods and apparatus for intraluminally or transluminally closing a left atrial appendage while under direct visualization are described herein. Such a system may include a deployment catheter and an attached imaging hood deployable into an expanded configuration. In use, the imaging hood is placed against or adjacent to a region of tissue to be imaged in a body lumen that is normally filled with an opaque bodily fluid such as blood. A translucent or transparent fluid, such as saline, can be pumped into the imaging hood until the fluid displaces any blood, thereby leaving a clear region of tissue to be imaged via an imaging element in the deployment catheter. Additionally, any number of therapeutic tools can also be passed through the deployment catheter and into the imaging hood for performing any number of procedures on the tissue for accessing and closing the left atrial appendage.

### CROSS-REFERENCE TO RELATED APPLICATIONS

This application claims the benefit of priority to U.S. Provisional Patent Application No. 60/821,113 filed Aug. 1, 2006, which is incorporated herein by reference in its entirety.

### FIELD OF THE INVENTION

The present invention relates generally to medical devices used for accessing, visualizing, and/or treating regions of tissue within a body. More particularly, the present invention relates to methods and apparatus for accessing, treating, and closing a left atrial appendage within a patient heart.

### BACKGROUND OF THE INVENTION

Conventional devices for accessing and visualizing interior regions of a body lumen are known. For example, ultrasound devices have been used to produce images from within a body in vivo. Ultrasound has been used both with and without contrast agents, which typically enhance ultrasound-derived images.

Other conventional methods have utilized catheters or probes having position sensors deployed within the body lumen, such as the interior of a cardiac chamber. These types of positional sensors are typically used to determine the movement of a cardiac tissue surface or the electrical activity within the cardiac tissue. When a sufficient number of points have been sampled by the sensors, a “map” of the cardiac tissue may be generated.

Another conventional device utilizes an inflatable balloon which is typically introduced intravascularly in a deflated state and then inflated against the tissue region to be examined. Imaging is typically accomplished by an optical fiber or other apparatus such as electronic chips for viewing the tissue through the membrane(s) of the inflated balloon. Moreover, the balloon must generally be inflated for imaging. Other conventional balloons utilize a cavity or depression formed at a distal end of the inflated balloon. This cavity or depression is pressed against the tissue to be examined and is flushed with a clear fluid to provide a clear pathway through the blood.

However, such imaging balloons have many inherent disadvantages. For instance, such balloons generally require that the balloon be inflated to a relatively large size which may undesirably displace surrounding tissue and interfere with fine positioning of the imaging system against the tissue. Moreover, the working area created by such inflatable balloons are generally cramped and limited in size. Furthermore, inflated balloons may be susceptible to pressure changes in the surrounding fluid. For example, if the environment surrounding the inflated balloon undergoes pressure changes, e.g., during systolic and diastolic pressure cycles in a beating heart, the constant pressure change may affect the inflated balloon volume and its positioning to produce unsteady or undesirable conditions for optimal tissue imaging.

Accordingly, these types of imaging modalities are generally unable to provide desirable images useful for sufficient diagnosis and therapy of the endoluminal structure, due in part to factors such as dynamic forces generated by the natural movement of the heart. Moreover, anatomic structures within the body can occlude or obstruct the image acquisition process. Also, the presence and movement of opaque bodily fluids such as blood generally make in vivo imaging of tissue regions within the heart difficult.

Other external imaging modalities are also conventionally utilized. For example, computed tomography (CT) and magnetic resonance imaging (MRI) are typical modalities which are widely used to obtain images of body lumens such as the interior chambers of the heart. However, such imaging modalities fail to provide real-time imaging for intra-operative therapeutic procedures. Fluoroscopic imaging, for instance, is widely used to identify anatomic landmarks within the heart and other regions of the body. However, fluoroscopy fails to provide an accurate image of the tissue quality or surface and also fails to provide for instrumentation for performing tissue manipulation or other therapeutic procedures upon the visualized tissue regions. In addition, fluoroscopy provides a shadow of the intervening tissue onto a plate or sensor when it may be desirable to view the intraluminal surface of the tissue to diagnose pathologies or to perform some form of therapy on it.

Moreover, many of the conventional imaging systems lack the capability to provide therapeutic treatments or are difficult to manipulate in providing effective therapies. For instance, treatment of a patient's heart for closing a left atrial appendage is one therapy which has been difficult. The LAA is a cavity connected to a lateral wall of the left atrium typically between the mitral valve and the left pulmonary vein. The LAA typically contracts with the left atrium which keeps blood from becoming stagnant. However, in many patients who experience conditions such as atrial fibrillation, the LAA may fail to contract often resulting in stagnant blood within the LAA and the subsequent formation of thrombus. Studies have suggested that the containment or removal of thrombus within the LAA in patients with atrial fibrillation may reduce the incidence of stroke. Access and closure of a LAA is generally made difficult by a number of factors, such as visualization of the target tissue, access to the target tissue, and instrument articulation and management, amongst others.

Thus, a tissue imaging system which is able to provide real-time in vivo access to and images of tissue regions within body lumens such as the heart through opaque media such as blood and which also provide instruments for therapeutic procedures upon the visualized tissue are desirable.

### SUMMARY OF THE INVENTION

A tissue imaging and manipulation apparatus that may be utilized for procedures within a body lumen, such as the heart, in which visualization of the surrounding tissue is made difficult, if not impossible, by medium contained within the lumen such as blood, is described below. Generally, such a tissue imaging and manipulation apparatus comprises an optional delivery catheter or sheath through which a deployment catheter and imaging hood may be advanced for placement against or adjacent to the tissue to be imaged.

The deployment catheter may define a fluid delivery lumen therethrough as well as an imaging lumen within which an optical imaging fiber or assembly may be disposed for imaging tissue. When deployed, the imaging hood may be expanded into any number of shapes, e.g., cylindrical, conical as shown, semi-spherical, etc., provided that an open area or field is defined by the imaging hood. The open area is the area within which the tissue region of interest may be imaged. The imaging hood may also define an atraumatic contact lip or edge for placement or abutment against the tissue region of interest. Moreover, the distal end of the deployment catheter or separate manipulatable catheters may be articulated through various controlling mechanisms such as push-pull wires manually or via computer control

In operation, after the imaging hood has been deployed, fluid may be pumped at a positive pressure through the fluid delivery lumen until the fluid fills the open area completely and displaces any blood from within the open area. The fluid may comprise any biocompatible fluid, e.g., saline, water, plasma, Fluorinert™, etc., which is sufficiently transparent to allow for relatively undistorted visualization through the fluid. The fluid may be pumped continuously or intermittently to allow for image capture by an optional processor which may be in communication with the assembly.

Once the imaging hood has been advanced into the left atrium of the heart, it may be articulated into apposition against the opening of a left atrial appendage (LAA). Once suitably positioned, the imaging hood and the cavity of the left atrial appendage may be purged with the transparent displacing fluid such that the tissue region and cavity may be visualized. Any number of procedures may be effected through the hood, such as delivery of an implant or adhesives into the left atrial appendage cavity. Alternatively, closure of the opening to the left atrial appendage may be accomplished intravascularly by the deployment of one or more tissue anchors connected via one or more lengths of suture or wire.

### BRIEF DESCRIPTION OF THE DRAWING

FIG. 1A shows a side view of one variation of a tissue imaging apparatus during deployment from a sheath or delivery catheter.

FIG. 1B shows the deployed tissue imaging apparatus of FIG. 1A having an optionally expandable hood or sheath attached to an imaging and/or diagnostic catheter.

FIG. 1C shows an end view of a deployed imaging apparatus.

FIGS. 1D to 1F show the apparatus of FIGS. 1A to 1C with an additional lumen, e.g., for passage of a guidewire therethrough.

FIGS. 2A and 2B show one example of a deployed tissue imager positioned against or adjacent to the tissue to be imaged and a flow of fluid, such as saline, displacing blood from within the expandable hood.

FIG. 3A shows an articulatable imaging assembly which may be manipulated via push-pull wires or by computer control.

FIGS. 3B and 3C show steerable instruments, respectively, where an articulatable delivery catheter may be steered within the imaging hood or a distal portion of the deployment catheter itself may be steered.

FIGS. 4A to 4C show side and cross-sectional end views, respectively, of another variation having an off-axis imaging capability.

FIGS. 5A and 5B show examples of various visualization imagers which may be utilized within or along the imaging hood.

FIGS. 6A to 6C illustrate deployment catheters having one or more optional inflatable balloons or anchors for stabilizing the device during a procedure.

FIGS. 7A and 7B illustrate a variation of an anchoring mechanism such as a helical tissue piercing device for temporarily stabilizing the imaging hood relative to a tissue surface.

FIG. 7C shows another variation for anchoring the imaging hood having one or more tubular support members integrated with the imaging hood; each support members may define a lumen therethrough for advancing a helical tissue anchor within.

FIG. 8A shows an illustrative example of one variation of how a tissue imager may be utilized with an imaging device.

FIG. 8B shows a further illustration of a hand-held variation of the fluid delivery and tissue manipulation system.

FIGS. 9A to 9C illustrate an example of capturing several images of the tissue at multiple regions.

FIGS. 10A and 10B show side views of a tissue visualization catheter with fluid flow enabling visualization on the LAA while implanting a number of closure devices.

FIG. 11 shows a side view of the tissue visualization catheter in another variation with an elongate bypass member extending from the hood and into the LAA to supply fluid therein.

FIGS. 12A to 12C show side views of the tissue visualization catheter delivering tissue anchors on the exterior tissue surface of the LAA under direct visualization such that the tissue anchors may be approximated to close the LAA.

FIGS. 13A to 13C show side views of the tissue visualization catheter delivering tissue anchors on the interior tissue surface of the LAA under direct visualization such that the tissue anchors may be approximated to close the LAA.

FIGS. 14A to 14C show side views of the tissue visualization catheter delivering helical tissue anchors on the interior tissue surface of the LAA under direct visualization such that the tissue anchors may be approximated to close the LAA.

FIGS. 15A to 15C show side views of the tissue visualization catheter drawing portions of the interior tissue surface of the LAA under direct visualization into a securement catheter to secure closure of the LAA.

FIG. 15D shows a perspective view of an example of a LAA closure staple.

FIGS. 16A and 16B show side and perspective views, respectively, of the tissue visualization catheter inserting an ablation probe between the tissue folds of a closed LAA.

FIG. 16C shows a side view of the LAA closure site with the ablated and/or scarred tissue in contact with each other after the ablation probe is retracted proximally into the visualization catheter.

FIGS. 17A and 17B show side views of the tissue visualization catheter having a suction catheter inserted into an enclosed LAA to suction the interior volume and/or to also inject an adhesive.

FIGS. 18A to 18C illustrate another variation where portions of the interior tissue surface of the LAA may be raised and ablated and subsequently adhered against one another to facilitate healing and closure of the tissue.

### DETAILED DESCRIPTION OF THE INVENTION

A tissue-imaging and manipulation apparatus described below is able to provide real-time images in vivo of tissue regions within a body lumen such as a heart, which is filled with blood flowing dynamically therethrough and is also able to provide intravascular tools and instruments for performing various procedures upon the imaged tissue regions. Such an apparatus may be utilized for many procedures, e.g., facilitating trans-septal access to the left atrium, cannulating the coronary sinus, diagnosis of valve regurgitation/stenosis, valvuloplasty, atrial appendage closure, arrhythmogenic focus ablation, among other procedures. Details of tissue imaging and manipulation systems and methods which may be utilized with apparatus and methods described herein are described in U.S. patent application Ser. No. 11/259,498 filed Oct. 25, 2005 (U.S. Pat. Pub. No. 2006/0184048 A1), which is incorporated herein by reference in its entirety.

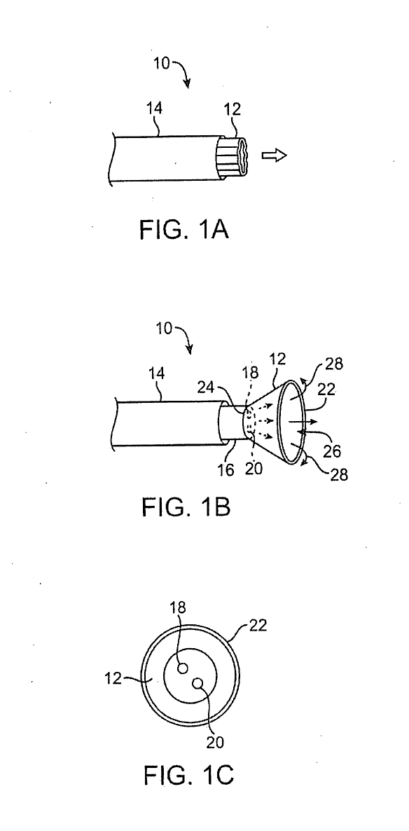

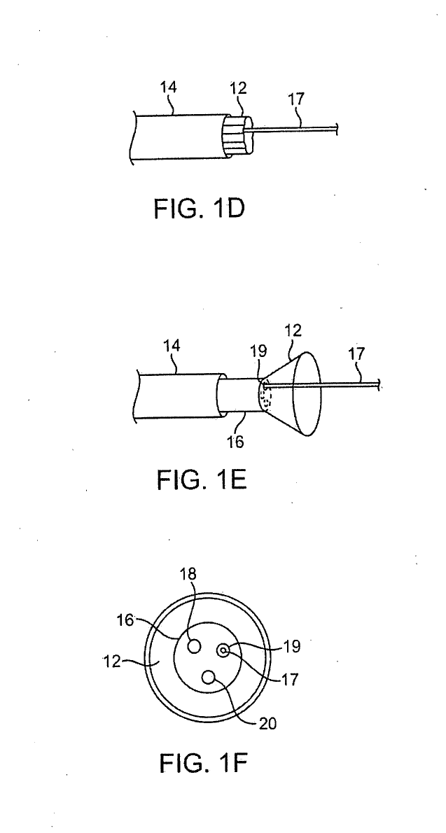

One variation of a tissue access and imaging apparatus is shown in the detail perspective views of FIGS. 1A to 1C. As shown in FIG. 1A, tissue imaging and manipulation assembly 10 may be delivered intravascularly through the patient's body in a low-profile configuration via a delivery catheter or sheath 14. In the case of treating tissue, such as the mitral valve located at the outflow tract of the left atrium of the heart, it is generally desirable to enter or access the left atrium while minimizing trauma to the patient. To non-operatively effect such access, one conventional approach involves puncturing the intra-atrial septum from the right atrial chamber to the left atrial chamber in a procedure commonly called a trans-septal procedure or septostomy. For procedures such as percutaneous valve repair and replacement, trans-septal access to the left atrial chamber of the heart may allow for larger devices to be introduced into the venous system than can generally be introduced percutaneously into the arterial system.

When the imaging and manipulation assembly 10 is ready to be utilized for imaging tissue, imaging hood 12 may be advanced relative to catheter 14 and deployed from a distal opening of catheter 14, as shown by the arrow. Upon deployment, imaging hood 12 may be unconstrained to expand or open into a deployed imaging configuration, as shown in FIG. 1B. Imaging hood 12 may be fabricated from a variety of pliable or conformable biocompatible material including but not limited to, e.g., polymeric, plastic, or woven materials. One example of a woven material is Kevlar® (E. I. du Pont de Nemours, Wilmington, Del.), which is an aramid and which can be made into thin, e.g., less than 0.001 in., materials which maintain enough integrity for such applications described herein. Moreover, the imaging hood 12 may be fabricated from a translucent or opaque material and in a variety of different colors to optimize or attenuate any reflected lighting from surrounding fluids or structures, i.e., anatomical or mechanical structures or instruments. In either case, imaging hood 12 may be fabricated into a uniform structure or a scaffold-supported structure, in which case a scaffold made of a shape memory alloy, such as Nitinol, or a spring steel, or plastic, etc., maybe fabricated and covered with the polymeric, plastic, or woven material.

Imaging hood 12 may be attached at interface 24 to a deployment catheter 16 which may be translated independently of deployment catheter or sheath 14. Attachment of interface 24 may be accomplished through any number of conventional methods. Deployment catheter 16 may define a fluid delivery lumen 18 as well as an imaging lumen 20 within which an optical imaging fiber or assembly may be disposed for imaging tissue. When deployed, imaging hood 12 may expand into any number of shapes, e.g., cylindrical, conical as shown, semi-spherical, etc., provided that an open area or field 26 is defined by imaging hood 12. The open area 26 is the area within which the tissue region of interest may be imaged. Imaging hood 12 may also define an atraumatic contact lip or edge 22 for placement or abutment against the tissue region of interest. Moreover, the diameter of imaging hood 12 at its maximum fully deployed diameter, e.g., at contact lip or edge 22, is typically greater relative to a diameter of the deployment catheter 16 (although a diameter of contact lip or edge 22 may be made to have a smaller or equal diameter of deployment catheter 16). For instance, the contact edge diameter may range anywhere from 1 to 5 times (or even greater, as practicable) a diameter of deployment catheter 16. FIG. 1C shows an end view of the imaging hood 12 in its deployed configuration. Also shown are the contact lip or edge 22 and fluid delivery lumen 18 and imaging lumen 20.

The imaging and manipulation assembly 10 may additionally define a guidewire lumen therethrough, e.g., a concentric or eccentric lumen, as shown in the side and end views, respectively, of FIGS. 1D to 1F. The deployment catheter 16 may define guidewire lumen 19 for facilitating the passage of the system over or along a guidewire 17, which may be advanced intravascularly within a body lumen. The deployment catheter 16 may then be advanced over the guidewire 17, as generally known in the art.

In operation, after imaging hood 12 has been deployed, as in FIG. 1B, and desirably positioned against the tissue region to be imaged along contact edge 22, the displacing fluid may be pumped at positive pressure through fluid delivery lumen 18 until the fluid fills open area 26 completely and displaces any fluid 28 from within open area 26. The displacing fluid flow may be laminarized to improve its clearing effect and to help prevent blood from re-entering the imaging hood 12. Alternatively, fluid flow may be started before the deployment takes place. The displacing fluid, also described herein as imaging fluid, may comprise any biocompatible fluid, e.g., saline, water, plasma, etc., which is sufficiently transparent to allow for relatively undistorted visualization through the fluid. Alternatively or additionally, any number of therapeutic drugs may be suspended within the fluid or may comprise the fluid itself which is pumped into open area 26 and which is subsequently passed into and through the heart and the patient body.

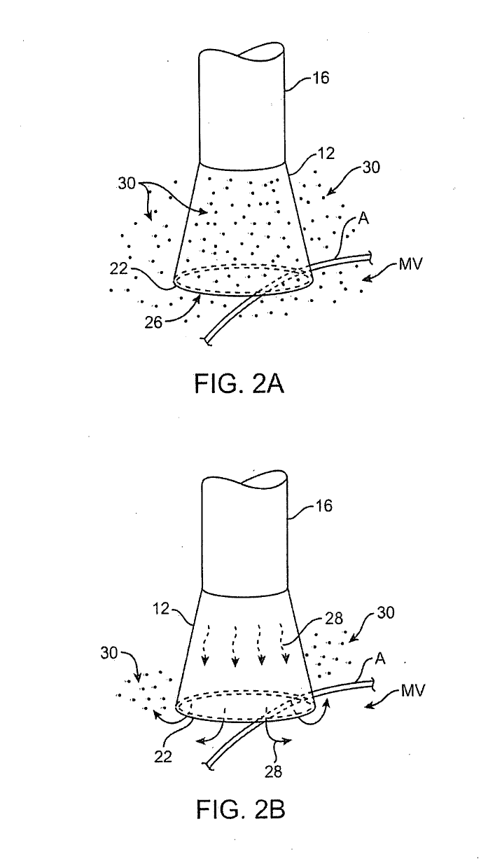

As seen in the example of FIGS. 2A and 2B, deployment catheter 16 may be manipulated to position deployed imaging hood 12 against or near the underlying tissue region of interest to be imaged, in this example a portion of annulus A of mitral valve MV within the left atrial chamber. As the surrounding blood 30 flows around imaging hood 12 and within open area 26 defined within imaging hood 12, as seen in FIG. 2A, the underlying annulus A is obstructed by the opaque blood 30 and is difficult to view through the imaging lumen 20. The translucent fluid 28, such as saline, may then be pumped through fluid delivery lumen 18, intermittently or continuously, until the blood 30 is at least partially, and preferably completely, displaced from within open area 26 by fluid 28, as shown in FIG. 2B.

Although contact edge 22 need not directly contact the underlying tissue, it is at least preferably brought into close proximity to the tissue such that the flow of clear fluid 28 from open area 26 may be maintained to inhibit significant backflow of blood 30 back into open area 26. Contact edge 22 may also be made of a soft elastomeric material such as certain soft grades of silicone or polyurethane, as typically known, to help contact edge 22 conform to an uneven or rough underlying anatomical tissue surface. Once the blood 30 has been displaced from imaging hood 12, an image may then be viewed of the underlying tissue through the clear fluid 30. This image may then be recorded or available for real-time viewing for performing a therapeutic procedure. The positive flow of fluid 28 may be maintained continuously to provide for clear viewing of the underlying tissue. Alternatively, the fluid 28 may be pumped temporarily or sporadically only until a clear view of the tissue is available to be imaged and recorded, at which point the fluid flow 28 may cease and blood 30 may be allowed to seep or flow back into imaging hood 12. This process may be repeated a number of times at the same tissue region or at multiple tissue regions.

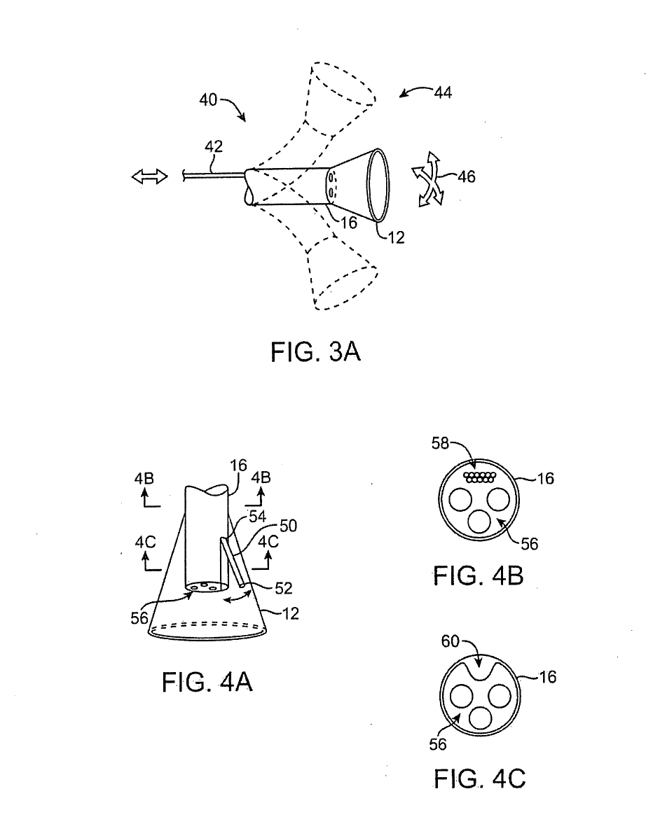

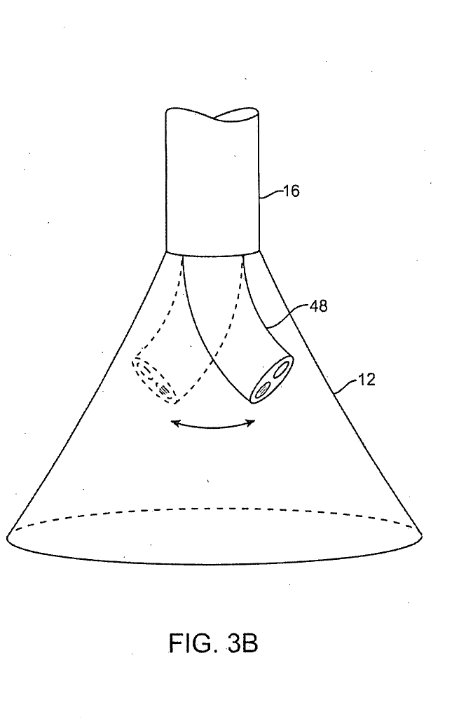

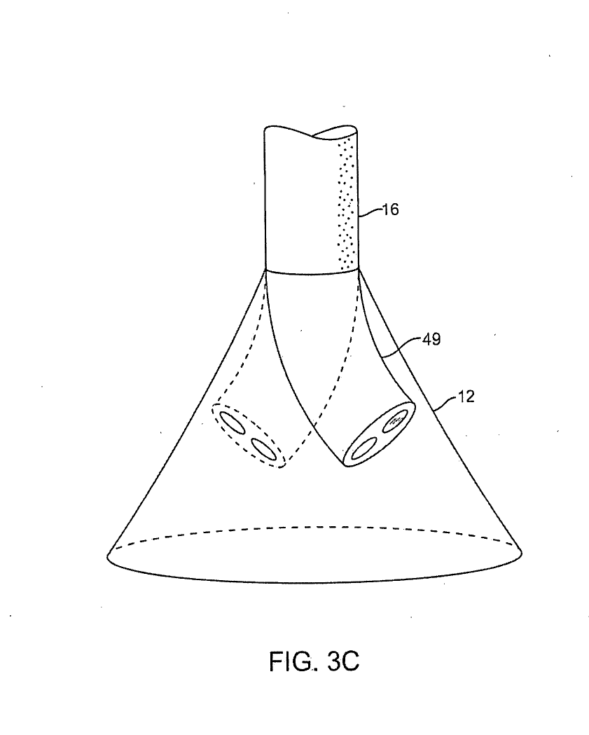

In desirably positioning the assembly at various regions within the patient body, a number of articulation and manipulation controls may be utilized. For example, as shown in the articulatable imaging assembly 40 in FIG. 3A, one or more push-pull wires 42 may be routed through deployment catheter 16 for steering the distal end portion of the device in various directions 46 to desirably position the imaging hood 12 adjacent to a region of tissue to be visualized. Depending upon the positioning and the number of push-pull wires 42 utilized, deployment catheter 16 and imaging hood 12 may be articulated into any number of configurations 44. The push-pull wire or wires 42 may be articulated via their proximal ends from outside the patient body manually utilizing one or more controls. Alternatively, deployment catheter 16 may be articulated by computer control, as further described below.

Additionally or alternatively, an articulatable delivery catheter 48, which may be articulated via one or more push-pull wires and having an imaging lumen and one or more working lumens, may be delivered through the deployment catheter 16 and into imaging hood 12. With a distal portion of articulatable delivery catheter 48 within imaging hood 12, the clear displacing fluid may be pumped through delivery catheter 48 or deployment catheter 16 to clear the field within imaging hood 12. As shown in FIG. 3B, the articulatable delivery catheter 48 may be articulated within the imaging hood to obtain a better image of tissue adjacent to the imaging hood 12. Moreover, articulatable delivery catheter 48 may be articulated to direct an instrument or tool passed through the catheter 48, as described in detail below, to specific areas of tissue imaged through imaging hood 12 without having to reposition deployment catheter 16 and re-clear the imaging field within hood 12.

Alternatively, rather than passing an articulatable delivery catheter 48 through the deployment catheter 16, a distal portion of the deployment catheter 16 itself may comprise a distal end 49 which is articulatable within imaging hood 12, as shown in FIG. 3C. Directed imaging, instrument delivery, etc., may be accomplished directly through one or more lumens within deployment catheter 16 to specific regions of the underlying tissue imaged within imaging hood 12.

Visualization within the imaging hood 12 may be accomplished through an imaging lumen 20 defined through deployment catheter 16, as described above. In such a configuration, visualization is available in a straight-line manner, i.e., images are generated from the field distally along a longitudinal axis defined by the deployment catheter 16. Alternatively or additionally, an articulatable imaging assembly having a pivotable support member 50 may be connected to, mounted to, or otherwise passed through deployment catheter 16 to provide for visualization off-axis relative to the longitudinal axis defined by deployment catheter 16, as shown in FIG. 4A. Support member 50 may have an imaging element 52, e.g., a CCD or CMOS imager or optical fiber, attached at its distal end with its proximal end connected to deployment catheter 16 via a pivoting connection 54.

If one or more optical fibers are utilized for imaging, the optical fibers 58 may be passed through deployment catheter 16, as shown in the cross-section of FIG. 4B, and routed through the support member 50. The use of optical fibers 58 may provide for increased diameter sizes of the one or several lumens 56 through deployment catheter 16 for the passage of diagnostic and/or therapeutic tools therethrough. Alternatively, electronic chips, such as a charge coupled device (CCD) or a CMOS imager, which are typically known, may be utilized in place of the optical fibers 58, in which case the electronic imager may be positioned in the distal portion of the deployment catheter 16 with electric wires being routed proximally through the deployment catheter 16. Alternatively, the electronic imagers may be wirelessly coupled to a receiver for the wireless transmission of images. Additional optical fibers or light emitting diodes (LEDs) can be used to provide lighting for the image or operative theater, as described below in further detail. Support member 50 may be pivoted via connection 54 such that the member 50 can be positioned in a low-profile configuration within channel or groove 60 defined in a distal portion of catheter 16, as shown in the cross-section of FIG. 4C. During intravascular delivery of deployment catheter 16 through the patient body, support member 50 can be positioned within channel or groove 60 with imaging hood 12 also in its low-profile configuration. During visualization, imaging hood 12 may be expanded into its deployed configuration and support member 50 may be deployed into its off-axis configuration for imaging the tissue adjacent to hood 12, as in FIG. 4A. Other configurations for support member 50 for off-axis visualization may be utilized, as desired.

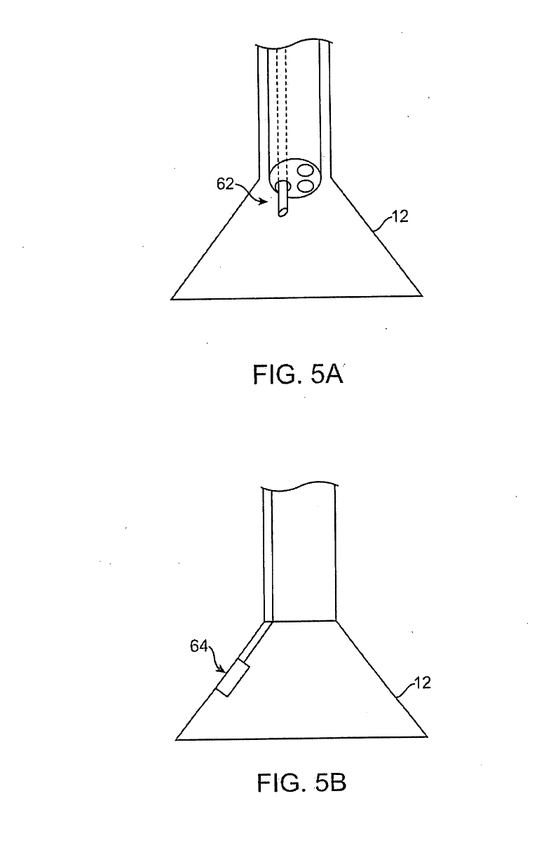

FIG. 5A shows a partial cross-sectional view of an example where one or more optical fiber bundles 62 may be positioned within the catheter and within imaging hood 12 to provide direct in-line imaging of the open area within hood 12. FIG. 5B shows another example where an imaging element 64 (e.g., CCD or CMOS electronic imager) may be placed along an interior surface of imaging hood 12 to provide imaging of the open area such that the imaging element 64 is off-axis relative to a longitudinal axis of the hood 12. The off-axis position of element 64 may provide for direct visualization and uninhibited access by instruments from the catheter to the underlying tissue during treatment.

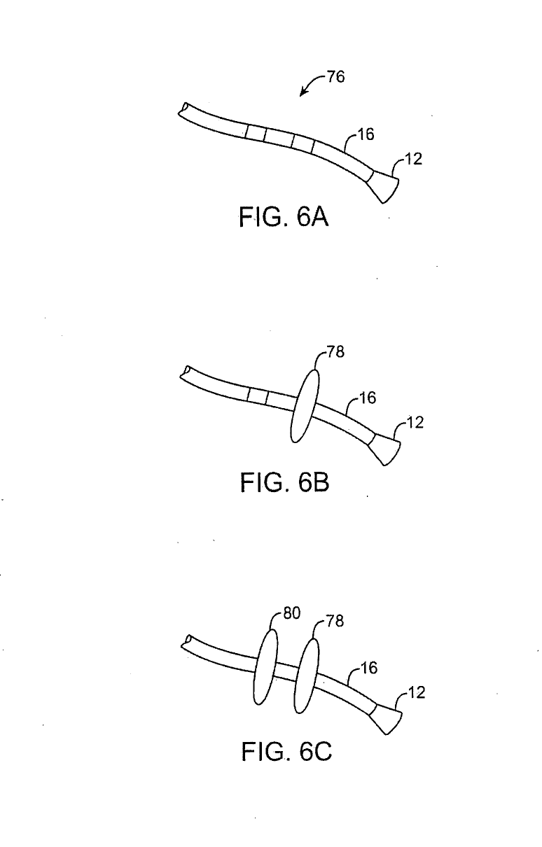

To facilitate stabilization of the deployment catheter 16 during a procedure, one or more inflatable balloons or anchors 76 may be positioned along the length of catheter 16, as shown in FIG. 6A. For example, when utilizing a trans-septal approach across the atrial septum AS into the left atrium LA, the inflatable balloons 76 may be inflated from a low-profile into their expanded configuration to temporarily anchor or stabilize the catheter 16 position relative to the heart H. FIG. 6B shows a first balloon 78 inflated while FIG. 6C also shows a second balloon 80 inflated proximal to the first balloon 78. In such a configuration, the septal wall AS may be wedged or sandwiched between the balloons 78, 80 to temporarily stabilize the catheter 16 and imaging hood 12. A single balloon 78 or both balloons 78, 80 may be used. Other alternatives may utilize expandable mesh members, malecots, or any other temporary expandable structure. After a procedure has been accomplished, the balloon assembly 76 may be deflated or re-configured into a low-profile for removal of the deployment catheter 16.

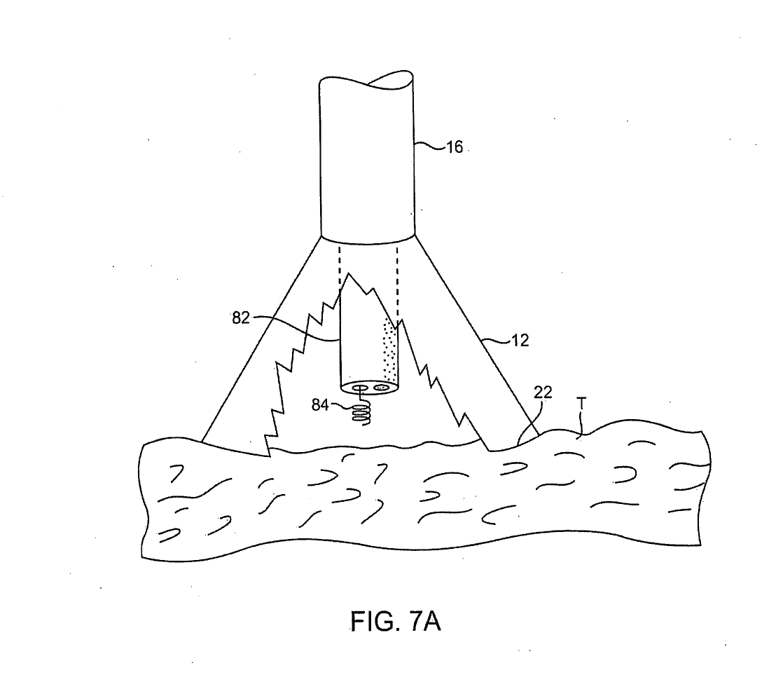

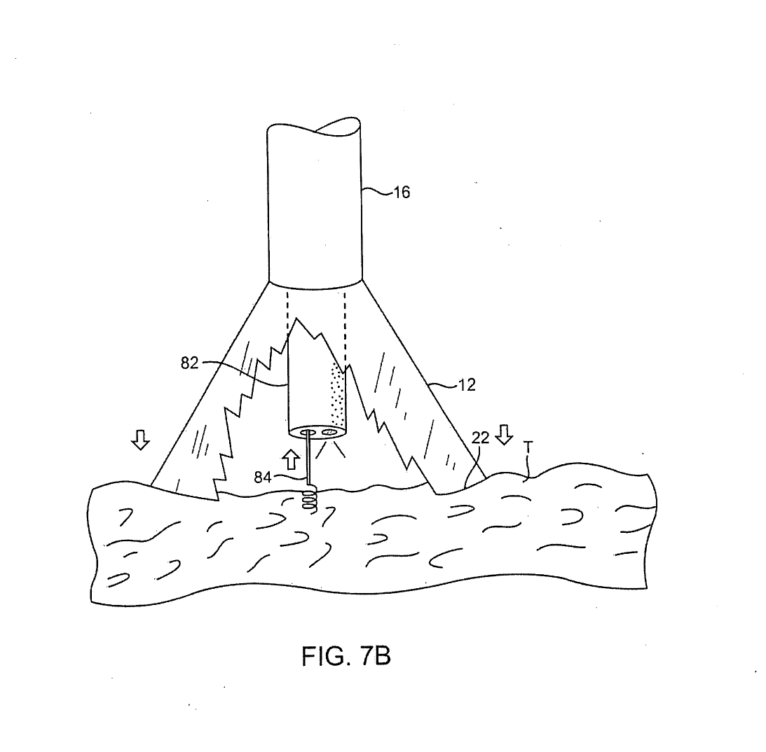

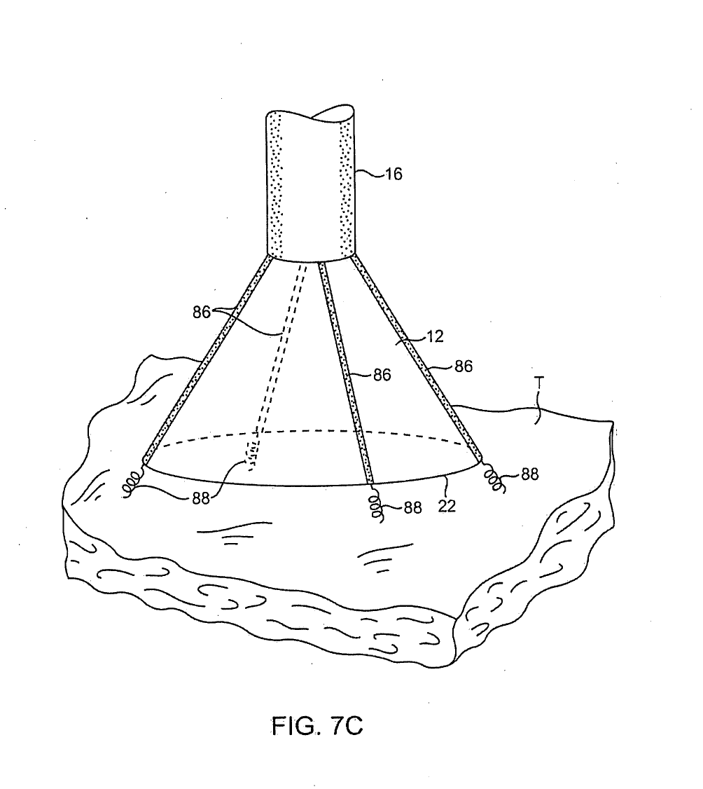

To further stabilize a position of the imaging hood 12 relative to a tissue surface to be imaged, various anchoring mechanisms may be optionally employed for temporarily holding the imaging hood 12 against the tissue. Such anchoring mechanisms may be particularly useful for imaging tissue which is subject to movement, e.g., when imaging tissue within the chambers of a beating heart. A tool delivery catheter 82 having at least one instrument lumen and an optional visualization lumen may be delivered through deployment catheter 16 and into an expanded imaging hood 12. As the imaging hood 12 is brought into contact against a tissue surface T to be examined, an anchoring mechanisms such as a helical tissue piercing device 84 may be passed through the tool delivery catheter 82, as shown in FIG. 7A, and into imaging hood 12.

The helical tissue engaging device 84 may be torqued from its proximal end outside the patient body to temporarily anchor itself into the underlying tissue surface T. Once embedded within the tissue T, the helical tissue engaging device 84 may be pulled proximally relative to deployment catheter 16 while the deployment catheter 16 and imaging hood 12 are pushed distally, as indicated by the arrows in FIG. 7B, to gently force the contact edge or lip 22 of imaging hood against the tissue T. The positioning of the tissue engaging device 84 may be locked temporarily relative to the deployment catheter 16 to ensure secure positioning of the imaging hood 12 during a diagnostic or therapeutic procedure within the imaging hood 12. After a procedure, tissue engaging device 84 may be disengaged from the tissue by torquing its proximal end in the opposite direction to remove the anchor form the tissue T and the deployment catheter 16 may be repositioned to another region of tissue where the anchoring process may be repeated or removed from the patient body. The tissue engaging device 84 may also be constructed from other known tissue engaging devices such as vacuum-assisted engagement or grasper-assisted engagement tools, among others.

Although a helical anchor 84 is shown, this is intended to be illustrative and other types of temporary anchors may be utilized, e.g., hooked or barbed anchors, graspers, etc. Moreover, the tool delivery catheter 82 may be omitted entirely and the anchoring device may be delivered directly through a lumen defined through the deployment catheter 16.

In another variation where the tool delivery catheter 82 may be omitted entirely to temporarily anchor imaging hood 12, FIG. 7C shows an imaging hood 12 having one or more tubular support members 86, e.g., four support members 86 as shown, integrated with the imaging hood 12. The tubular support members 86 may define lumens therethrough each having helical tissue engaging devices 88 positioned within. When an expanded imaging hood 12 is to be temporarily anchored to the tissue, the helical tissue engaging devices 88 may be urged distally to extend from imaging hood 12 and each may be torqued from its proximal end to engage the underlying tissue T. Each of the helical tissue engaging devices 88 may be advanced through the length of deployment catheter 16 or they may be positioned within tubular support members 86 during the delivery and deployment of imaging hood 12. Once the procedure within imaging hood 12 is finished, each of the tissue engaging devices 88 may be disengaged from the tissue and the imaging hood 12 may be repositioned to another region of tissue or removed from the patient body.

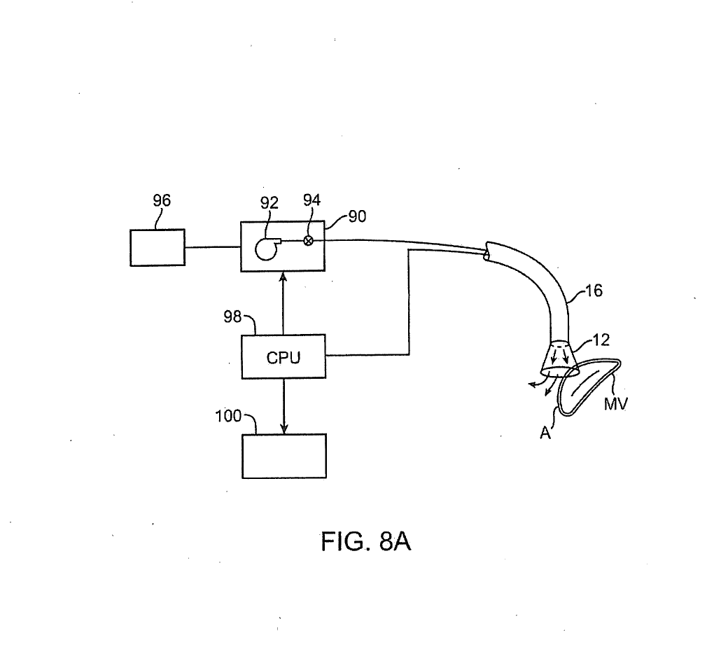

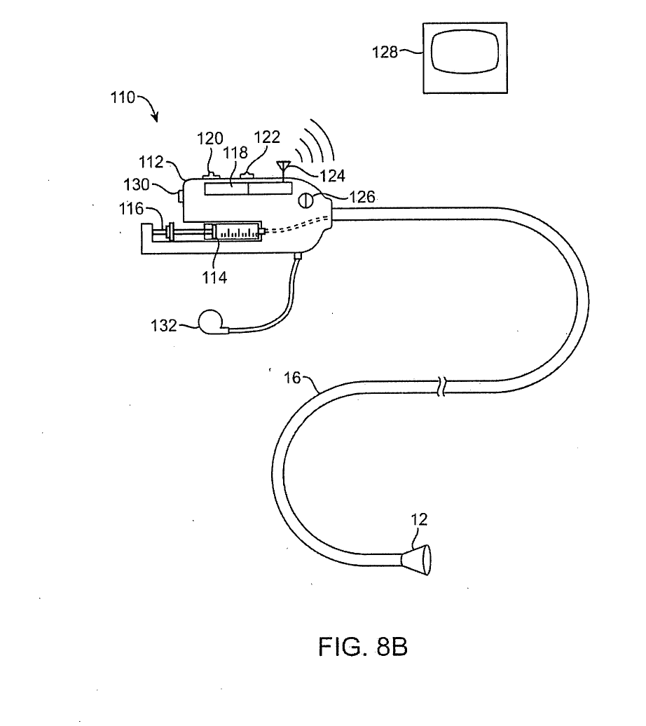

An illustrative example is shown in FIG. 8A of a tissue imaging assembly connected to a fluid delivery system 90 and to an optional processor 98 and image recorder and/or viewer 100. The fluid delivery system 90 may generally comprise a pump 92 and an optional valve 94 for controlling the flow rate of the fluid into the system. A fluid reservoir 96, fluidly connected to pump 92, may hold the fluid to be pumped through imaging hood 12. An optional central processing unit or processor 98 may be in electrical communication with fluid delivery system 90 for controlling flow parameters such as the flow rate and/or velocity of the pumped fluid. The processor 98 may also be in electrical communication with an image recorder and/or viewer 100 for directly viewing the images of tissue received from within imaging hood 12. Imager recorder and/or viewer 100 may also be used not only to record the image but also the location of the viewed tissue region, if so desired.

Optionally, processor 98 may also be utilized to coordinate the fluid flow and the image capture. For instance, processor 98 may be programmed to provide for fluid flow from reservoir 96 until the tissue area has been displaced of blood to obtain a clear image. Once the image has been determined to be sufficiently clear, either visually by a practitioner or by computer, an image of the tissue may be captured automatically by recorder 100 and pump 92 may be automatically stopped or slowed by processor 98 to cease the fluid flow into the patient. Other variations for fluid delivery and image capture are, of course, possible and the aforementioned configuration is intended only to be illustrative and not limiting.

FIG. 8B shows a further illustration of a hand-held variation of the fluid delivery and tissue manipulation system 110. In this variation, system 110 may have a housing or handle assembly 112 which can be held or manipulated by the physician from outside the patient body. The fluid reservoir 114, shown in this variation as a syringe, can be fluidly coupled to the handle assembly 112 and actuated via a pumping mechanism 116, e.g., lead screw. Fluid reservoir 114 maybe a simple reservoir separated from the handle assembly 112 and fluidly coupled to handle assembly 112 via one or more tubes. The fluid flow rate and other mechanisms may be metered by the electronic controller 118.

Deployment of imaging hood 12 may be actuated by a hood deployment switch 120 located on the handle assembly 112 while dispensation of the fluid from reservoir 114 may be actuated by a fluid deployment switch 122, which can be electrically coupled to the controller 118. Controller 118 may also be electrically coupled to a wired or wireless antenna 124 optionally integrated with the handle assembly 112, as shown in the figure. The wireless antenna 124 can be used to wirelessly transmit images captured from the imaging hood 12 to a receiver, e.g., via Bluetooth® wireless technology (Bluetooth SIG, Inc., Bellevue, Wash.), RF, etc., for viewing on a monitor 128 or for recording for later viewing.

Articulation control of the deployment catheter 16, or a delivery catheter or sheath 14 through which the deployment catheter 16 may be delivered, may be accomplished by computer control, as described above, in which case an additional controller may be utilized with handle assembly 112. In the case of manual articulation, handle assembly 112 may incorporate one or more articulation controls 126 for manual manipulation of the position of deployment catheter 16. Handle assembly 112 may also define one or more instrument ports 130 through which a number of intravascular tools may be passed for tissue manipulation and treatment within imaging hood 12, as described further below. Furthermore, in certain procedures, fluid or debris may be sucked into imaging hood 12 for evacuation from the patient body by optionally fluidly coupling a suction pump 132 to handle assembly 112 or directly to deployment catheter 16.

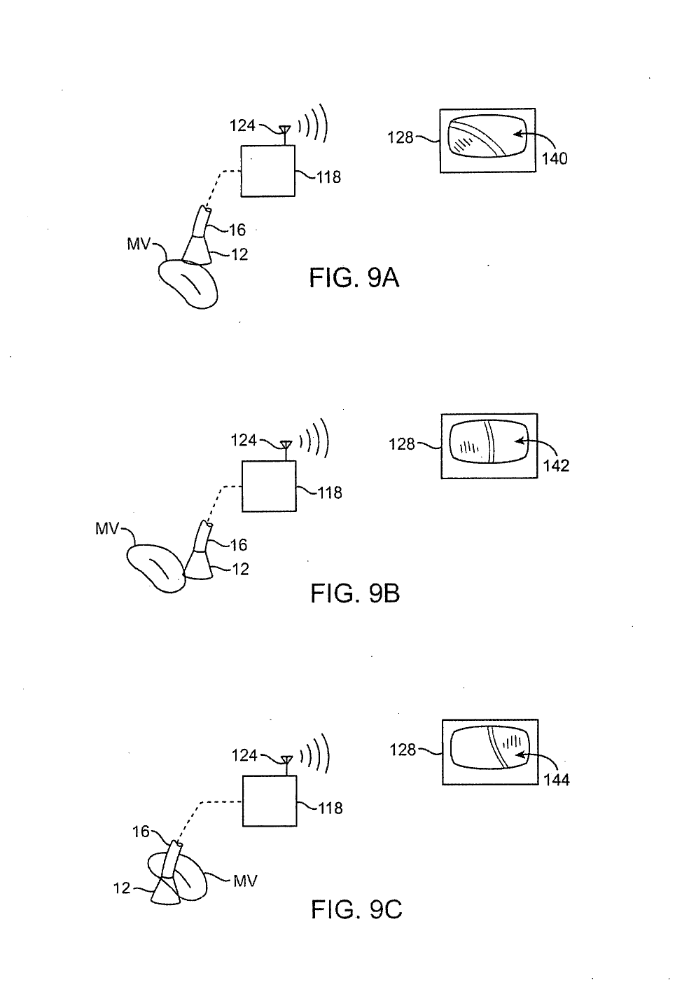

As described above, fluid may be pumped continuously into imaging hood 12 to provide for clear viewing of the underlying tissue. Alternatively, fluid may be pumped temporarily or sporadically only until a clear view of the tissue is available to be imaged and recorded, at which point the fluid flow may cease and the blood may be allowed to seep or flow back into imaging hood 12. FIGS. 9A to 9C illustrate an example of capturing several images of the tissue at multiple regions. Deployment catheter 16 may be desirably positioned and imaging hood 12 deployed and brought into position against a region of tissue to be imaged, in this example the tissue surrounding a mitral valve MV within the left atrium of a patient's heart. The imaging hood 12 may be optionally anchored to the tissue, as described above, and then cleared by pumping the imaging fluid into the hood 12. Once sufficiently clear, the tissue may be visualized and the image captured by control electronics 118. The first captured image 140 may be stored and/or transmitted wirelessly 124 to a monitor 128 for viewing by the physician, as shown in FIG. 9A.

The deployment catheter 16 may be then repositioned to an adjacent portion of mitral valve MV, as shown in FIG. 9B, where the process may be repeated to capture a second image 142 for viewing and/or recording. The deployment catheter 16 may again be repositioned to another region of tissue, as shown in FIG. 9C, where a third image 144 may be captured for viewing and/or recording. This procedure may be repeated as many times as necessary for capturing a comprehensive image of the tissue surrounding mitral valve MV, or any other tissue region. When the deployment catheter 16 and imaging hood 12 is repositioned from tissue region to tissue region, the pump may be stopped during positioning and blood or surrounding fluid may be allowed to enter within imaging hood 12 until the tissue is to be imaged, where the imaging hood 12 may be cleared, as above.

As mentioned above, when the imaging hood 12 is cleared by pumping the imaging fluid within for clearing the blood or other bodily fluid, the fluid may be pumped continuously to maintain the imaging fluid within the hood 12 at a positive pressure or it may be pumped under computer control for slowing or stopping the fluid flow into the hood 12 upon detection of various parameters or until a clear image of the underlying tissue is obtained. The control electronics 118 may also be programmed to coordinate the fluid flow into the imaging hood 12 with various physical parameters to maintain a clear image within imaging hood 12.

In utilizing the visualization assembly for procedures such as the intravascular closure of a left atrial appendage (LAA), the hood assembly may be advanced intravascularly into the right atrium of the patient's heart. The hood assembly may then be advanced transseptally into the left atrium LA, where it may then be articulated into contact against the LAA. Once a sufficient seal has been achieved between the hood and tissue surrounding the LAA opening, the transparent displacement fluid may be infused into the hood and the cavity of the LAA to enable direct visualization of the tissue structures. Detail examples and descriptions of a visualization catheter device and system which may be utilized herein are shown and described in further detail in U.S. patent application Ser. No. 11/259,498 filed Oct. 25, 2005, which has been incorporated herein above in its entirety, and further details of transseptal access methods and systems which may also be utilized herein are shown in Ser. No. 11/763,399 filed Jun. 14, 2007, which is incorporated herein by reference in its entirety.

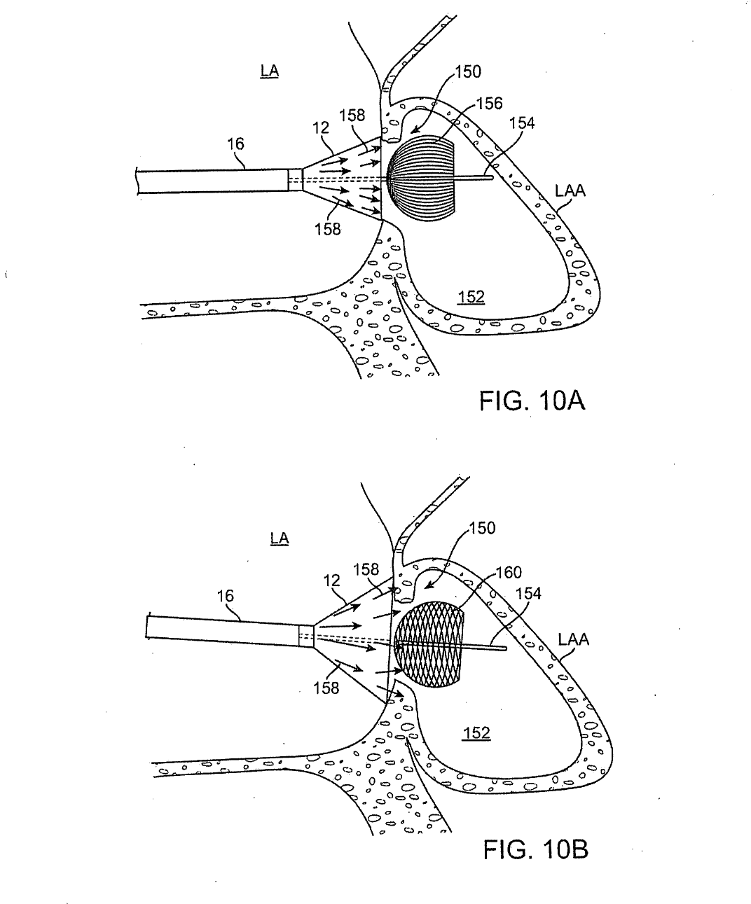

As shown in the partial cross-sectional side view of FIG. 10A, with hood 12 expanded and placed against LAA opening 150, the displacement fluid 158 may be infused into hood 12 and into LAA volume 152. An expandable closure device 156 may be delivered in a low profile configuration into the LAA along a guidewire or elongate member 154 advanced through catheter 16 and through hood 12 and into LAA, where it may then be expanded into an enlarged state, as shown. Closure device 156 may be configured to expand into various shapes, such as a disk or spherically-shaped scaffold or membrane. Once the closure device 156 has been deployed, it may be detached from the catheter device 16 to remain within the LAA to seal the LAA off from the atrial chamber.

FIG. 10B shows another variation where hood 12 is sized sufficiently-to ensure coverage of the entire LAA opening 150 to enable visualization of the entire LAA, including the opening 150. The use of an enlarged hood may also enable the capture and/or removal of blood clots which may be found in the LAA by inhibiting or preventing the release of blood clots or debris which may be dislodged from within the LAA. With a larger hood compassing the entire LAA opening 150, blood clots removed from the LAA may be captured by the hood and disposed through the catheter's irrigation channel. Additionally, this variation illustrates the use of an expandable mesh closure device 160 as another example of a closure device which maybe utilized to occlude the LAA opening 150.

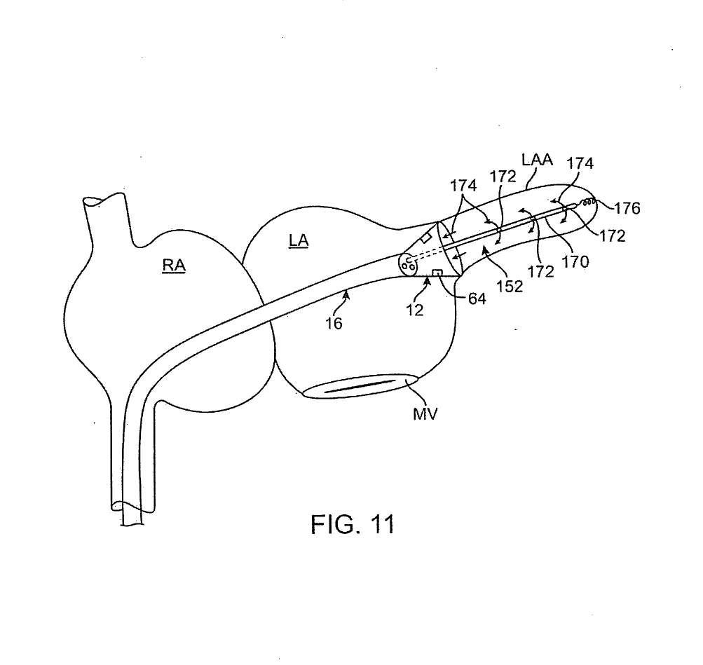

FIG. 11 illustrates another variation of an assembly with an elongate infusion catheter 170 which can be advanced at least partially into the LAA to infuse a displacement fluid 174 into the LAA. As described above, the catheter 16 and deployed hood 12 may be positioned over the LAA opening while infusion catheter 170 is advanced into the LAA cavity 152. Infusion catheter 170 may define a plurality of openings 172 along its outer surface through which the displacement fluid 174 may be infused into LAA cavity 152. A lumen in the deployment catheter 16 which is in fluid communication with the open area of hood 12 may evacuate the fluid 174 and may also aspirate blood located in the left atrial appendage LAA. A variation of the assembly may include a tissue attachment member 176, such as a helical tissue engager, positioned upon a distal end of infusion catheter 170 to stabilize the assembly with respect to the LAA opening. In use, once the infusion catheter 170 has been advanced into the LAA cavity 152, the tissue attachment member 176 may be rotated into the tissue to secure the hood 12 with respect to the LAA. As noted above, the infusion catheter 170 may be moveable relative to the deployment catheter 16 and hood 12; accordingly, pulling the infusion catheter 170 when the tissue attachment member 176 is attached to tissue may assist in creating a compressive force between the hood 12 and the opening of the LAA. With blood displaced from the LAA cavity 152 by saline discharged from the infusion catheter 170, direct real-time in vivo visualization of the entire LAA may be possible.

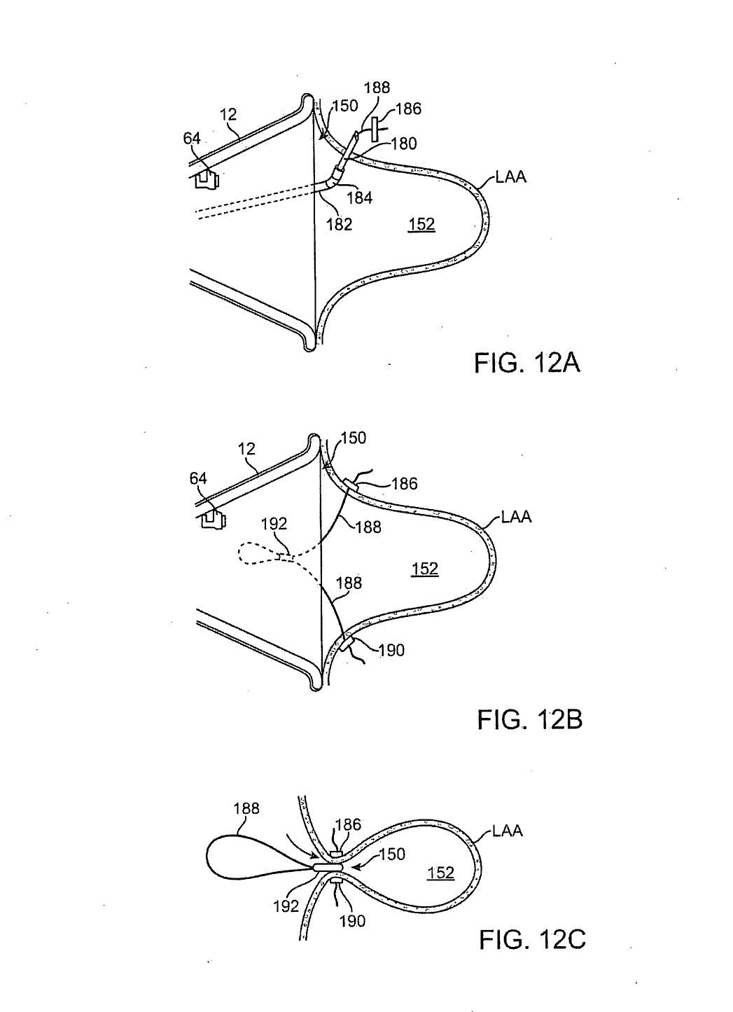

FIGS. 12A to 12C illustrate another method of closing an LAA by delivering anchors transluminally under direct visualization with the tissue visualization catheter platform. As shown in FIG. 12A, with hood 12 deployed and placed over the LAA opening 150, the LAA cavity 152 and hood 12 may be purged of blood with the transparent displacement fluid while under visualization from the imaging element 64. Thus, while viewed by the user, a needle body 180 disposed upon needle catheter 182 may be advanced through deployment catheter 16 and through hood 12 and articulated, e.g., via an optional pivot 184, to puncture the wall of the LAA. A first tissue anchor 186 connected via a length of wire or suture 188 and housed within the needle body 180 may be ejected to the exterior of the LAA. This process may be repeated along the LAA wall opposite to where the first tissue anchor 186 was deployed. At this second tissue region, a second tissue anchor 190 may also be similarly deployed along the exterior of the LAA, as shown in FIG. 12B. Each of the anchors 186, 190 may be interconnected via the length of wire or suture 188, which may have a slidable locking mechanism 192 disposed thereon.

The LAA may be closed upon pushing locking mechanism 192 along the length of the suture 188 towards the LAA opening 150. The locking mechanism 192 may be configured to slide in a unidirectional manner to approximate the pair of anchors 186, 190 towards one another and lock them in place relative to one another, as shown in FIG. 12C. This motion results in closure of the LAA opening 150. Various examples of cinching mechanisms are shown and described in further detail in U.S. Pat. No. 7,186,262 and U.S. Pat. Pub. 2004/0044364A1, each of which is incorporated herein by reference in its entirety. Additional anchors may be deployed around the circumference of the LAA opening 150 to ensure complete closure.

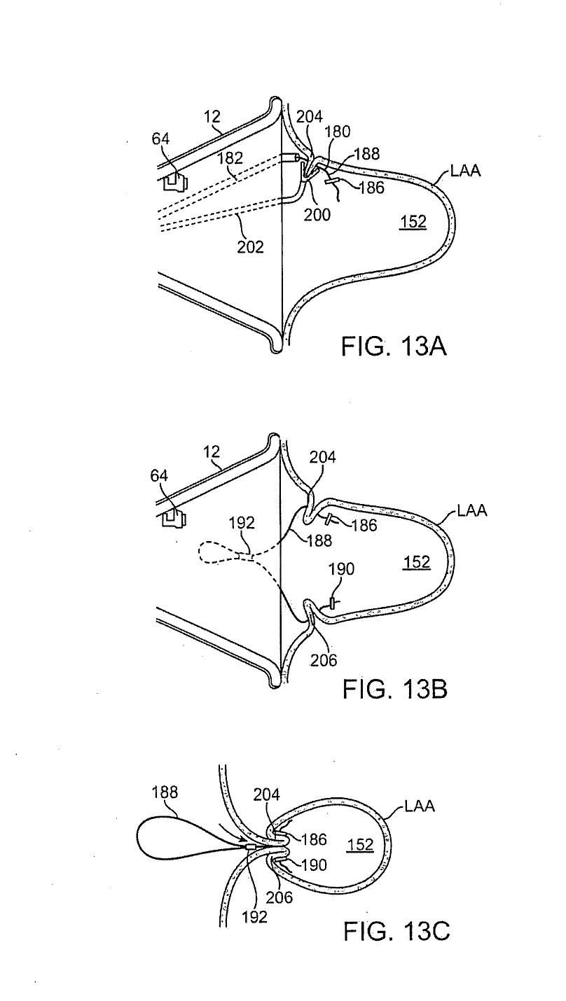

FIGS. 13A to 13C show another method of closing the LAA by delivering anchors intraluminally while also utilizing a tissue grasper 200 disposed upon a grasper catheter 202 passed through the deployment catheter 16 and hood 12. Grasper 200 may be used to create an intraluminal tissue fold 204 at a predetermined spot while under direct visualization from imaging element 64. While the grasper 200 maintains the tissue fold 204, needle body 180 may be passed through the fold such that first tissue anchor 186 may be urged from needle body 180. Grasper 200 and needle body 180 may be relocated to an opposite side of the LAA wall where the process may be repeated and a second tissue anchor 190 may be released intraluminally, as shown in FIG. 6B, through a second tissue fold 206. Both anchors 186, 190 may be approximated towards one another by the cinching of locking mechanism 192 along the length of wire or suture 188 interconnecting the two anchors to close the LAA opening 150. As above, this process may be repeated around the circumference of the LAA to ensure its closure.

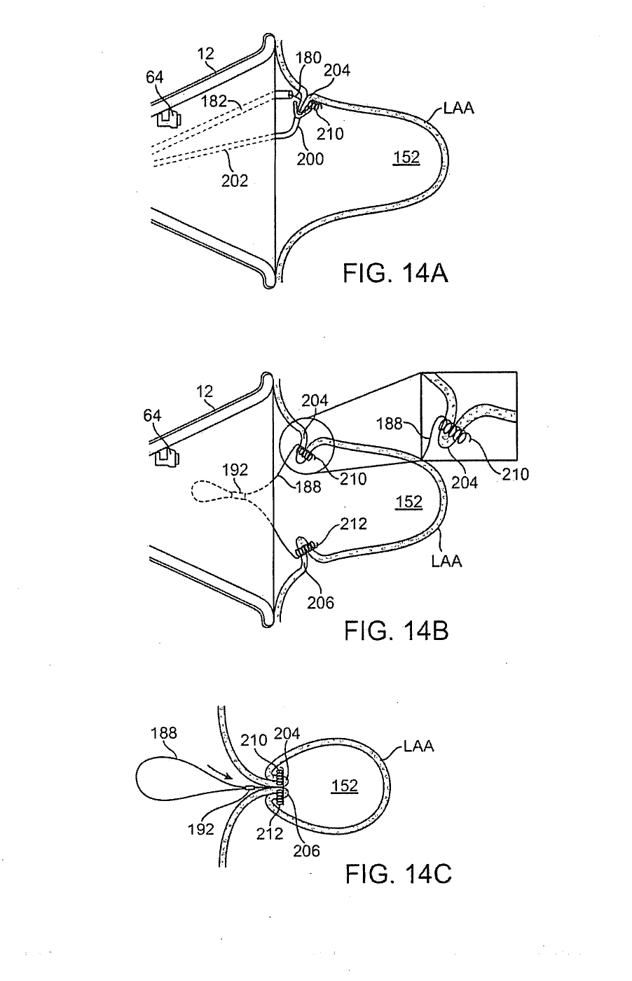

FIGS. 14A to 14C show yet another method of closing the LAA by delivering helical tissue grasping anchors without the need of a needle body. As above, tissue grasper 200 may form a first tissue fold 204 through which a first helical tissue anchor 210 may be attached by rotating along its longitudinal axis to allow the helical anchor 210 to penetrate and hold the target tissue in place. The process may be repeated through a second tissue fold 206 located on the LAA tissue wall opposite to the first tissue fold 204 where second helical tissue anchor 212 may be rotated into the tissue, as shown in FIG. 14B. Each of the anchors 210, 212 may be interconnected to one another and closure of the LAA opening 150 may be accomplished by the cinching of locking mechanism 192 along wire or suture 188 to approximate the anchors 210, 212, as described above and as shown in FIG. 14C.

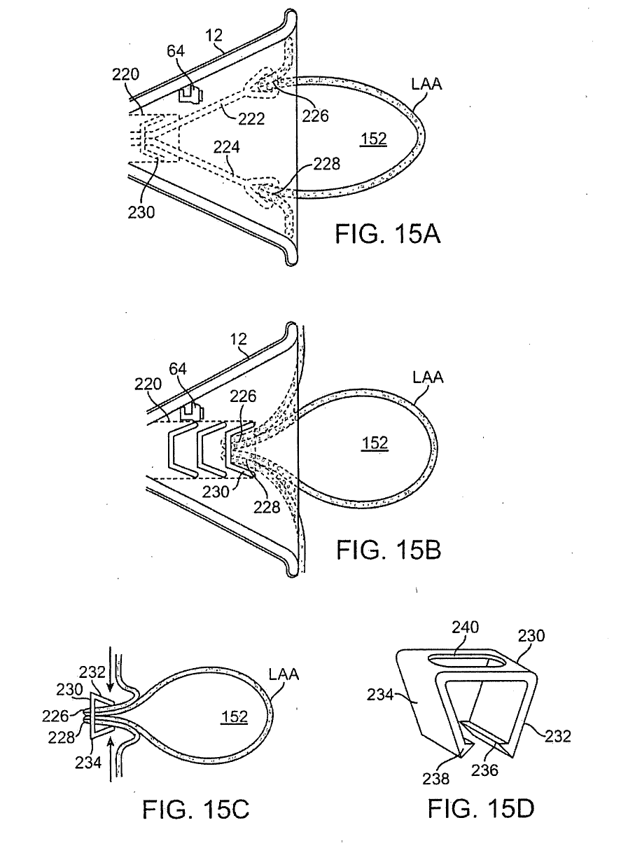

In yet another variation for closing the LAA, a pair of grasping members 222, 224 extending from a catheter 220 delivered through deployment catheter 16 and through hood 12 may be used to engage at least two apposing regions of tissue 226, 228 around the circumference of the LAA opening while under direct visualization from imaging element 64, as shown in FIG. 15A. First and second grasping members 222, 224 may be retracted proximally into hood 12 and into catheter 220 while maintaining a grasp on the respective folds of tissue 222, 224, where at least one tissue securement device 230 may be secured upon tissue folds 222, 224, as shown in FIG. 15B.

With the grasping members 222, 224 retracted, tissue securement device 230 may be clamped or stapled upon the approximated tissue by bringing a first and second securement arm 232, 234 of device 230 towards one another onto the approximated tissue. Alternatively, the clamping action may be achieved by configuring the arms 232, 234 to be biased towards one another such that when the grasping members 222, 224 are pulled proximally through an opening 240 defined through device 230, the unconstrained arms 232, 234 may spring towards one another to penetrate into the approximated tissue and subsequently generating an axial force inwardly on the tissue. The securement device 230 may comprise various staples, clamps, clips, or other tissue affixation mechanisms and may further define first and second tissue attachment features 236, 238, such as barbs as shown in FIG. 15D, to facilitate the adherence and securement of the device 230 onto the tissue.

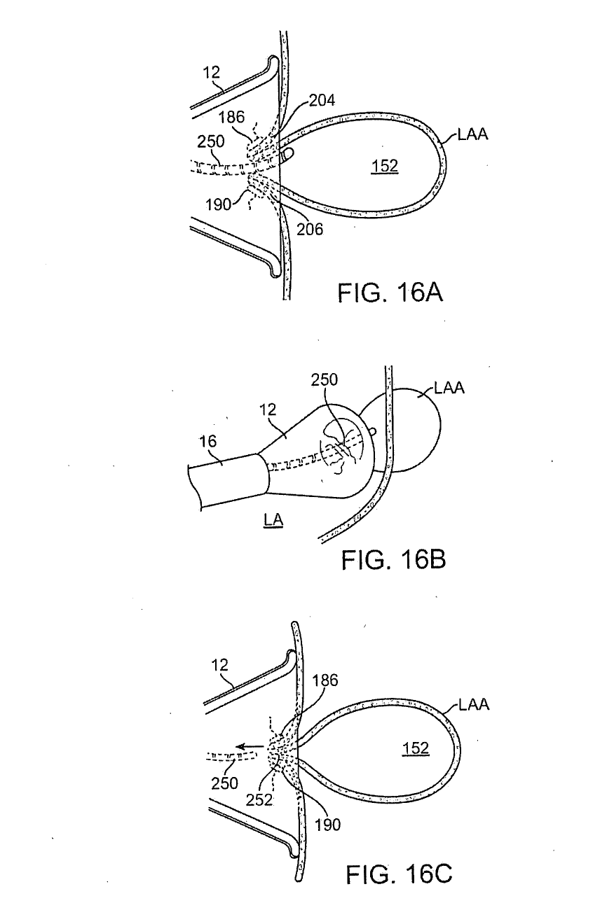

In closing or occluding the opening of the LAA, as described above, additional methods may be optionally utilized to enhance the closure of the LAA while under direct visualization from an imaging element. In one method, portions of the tissue surrounding the LAA which has been or is to be approximated together may be ablated or otherwise scarred to facilitate tissue adhesion upon healing. As shown in FIGS. 16A and 16B, an ablation probe 250 may be advanced through deployment catheter 16 and through hood 12 and then inserted through the “seam” of the closure site. The contacted tissue may be ablated, e.g., by RF, laser, HIFU, or microwave, etc., and the ablation probe 250 may be then retracted into the deployment catheter 16 leaving the ablated and/or scarred tissues 252 in contact with each other at the closure site, as shown in FIG. 16C. Upon healing, tissue adhesion will be possible between the scarred areas 252. This can act as a secondary or primary method to close or improve the sealing of LAA.

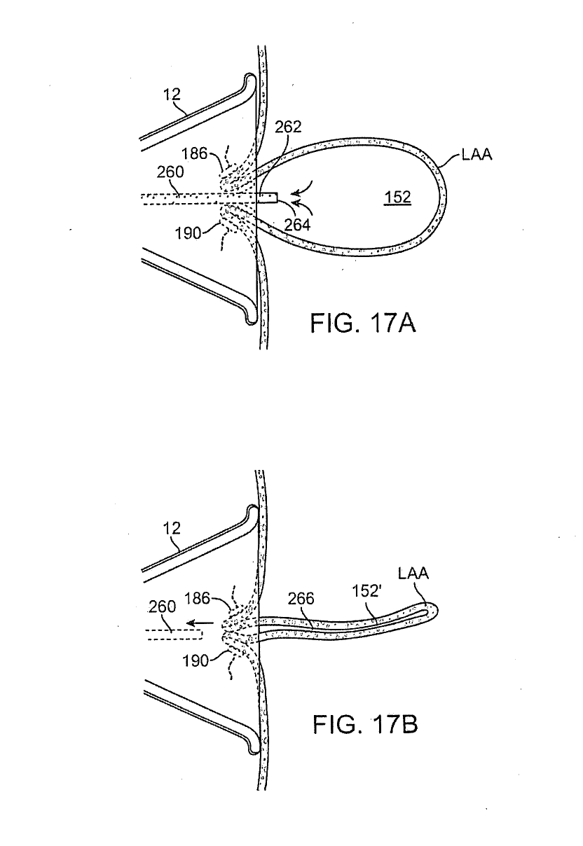

Another example for enhancing the closure or occlusion of an LAA cavity is shown in FIGS. 17A and 17B, which illustrate the application of a vacuum to deflate or collapse the LAA cavity and the introduction of a tissue adhesive to maintain the LAA in its collapsed state. As shown in FIG. 17A, suction catheter 260 having a suction opening 264 defined at its distal tip may be inserted through the “seam” of the closure site and activated to draw a suction force to deflate or collapse the LAA. Suction catheter 260 may also optionally define one or more ports or openings 262 along its outer surface through which a biocompatible adhesive or glue 266 may be injected into the collapsed LAA cavity 152′ to enhance sealing between the apposed and contacting layers of tissue, as shown in FIG. 17B.

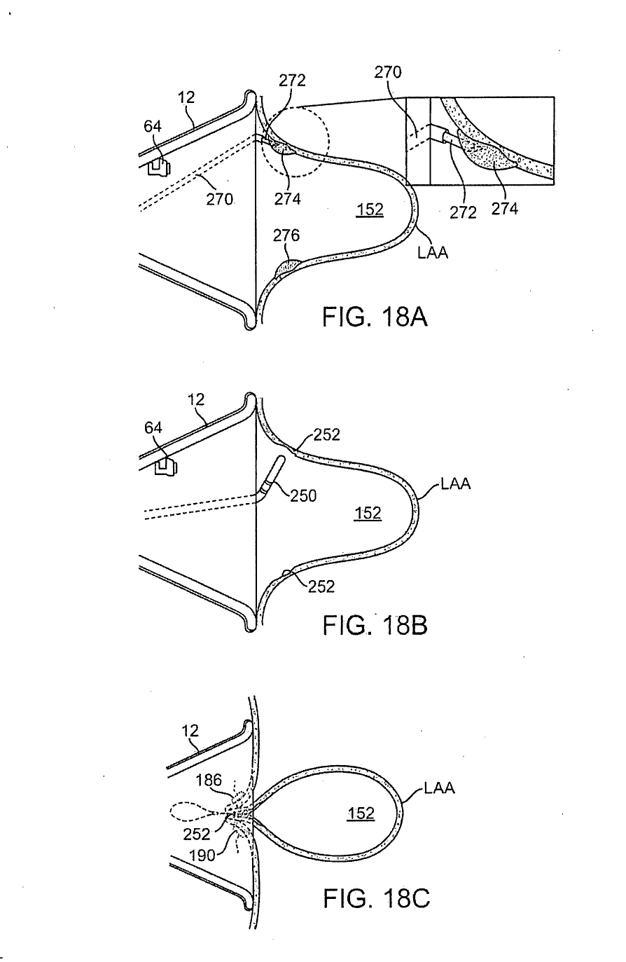

In yet another example for enhancing the closure of an LAA cavity, FIGS. 18A to 18C illustrate a variation where an infusion needle 272 disposed upon infusion catheter 270 may be advanced within hood 12 and into a portion of the tissue surrounding the LAA while under direct visualization from imaging element 64. Saline may be injected through needle 272 into the tissue to raise the tissue surface 274, 276 at one or more locations around the LAA to be approximated. With the tissue surfaces raised, an ablation probe 250 may be advanced into contact against the raised tissue surfaces 274, 276 to ablate the raised tissue without damage the underlying tissue structure, as shown in FIG. 18B. Once the appropriate tissue regions have been ablated, they may then be approximated into contact against one another, as described above, to enhance tissue adhesion during the healing process and to ensure closure of the LAA from the remainder of the atrial chamber.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A method for closing a left atrial appendage within a patient body, comprising:
intravascularly advancing a deployment catheter adjacent to an opening of a left atrial appendage;
positioning an expanded imaging hood projecting distally from the deployment catheter against or over the opening;
urging a transparent fluid into the hood via the deployment catheter such that an opaque fluid is displaced from the hood;
visualizing an interior cavity of the left atrial appendage through the translucent fluid; and
closing the opening of the left atrial appendage.

2. The method of claim 1 wherein intravascularly advancing a deployment catheter comprises advancing the catheter transseptally through an atrial septum and into a left atrial chamber of a heart.

3. The method of claim 1 wherein positioning an expanded imaging hood comprises deploying the hood from a low-profile delivery configuration within a sheath into an expanded deployed configuration external to the sheath.

4. The method of claim 1 wherein urging a transparent fluid comprises pumping the transparent fluid into the hood through a fluid delivery lumen defined through the deployment catheter.

5. The method of claim 4 wherein pumping the transparent fluid comprises urging saline, plasma, water, or perfluorinated liquid into the hood such that blood is displaced from the hood and the interior cavity of the left atrial appendage.

6. The method of claim 1 wherein closing the opening comprises deploying an expandable implant into the cavity such that the opening is occluded.

7. The method of claim 6 wherein deploying comprises expanding a mesh or scaffold structure from a low-profile configuration into an expanded configuration within the cavity.

8. The method of claim 1 wherein closing the opening comprises:
deploying at least one pair of tissue anchors connected by a length of wire or suture into a circumference of the opening; and
approximating the pair of tissue anchors towards one another such that the opening is closed.

9. The method of claim 8 wherein deploying comprises ejecting the at least one pair of tissue anchors along an exterior tissue surface of the left atrial appendage.

10. The method of claim 8 wherein deploying comprises passing the at least one pair of tissue anchors through a respective tissue fold within the left atrial appendage.

11. The method of claim 8 wherein approximating comprises cinching a locking mechanism along the wire or suture such that a relative position of the tissue anchors are inhibited from movement with respect to one another.

12. The method of claim 1 further comprising damaging tissue around the opening in contact with one another.

13. The method of claim 12 wherein damaging tissue comprises ablating or scarring the tissue via an ablation probe.

14. The method of claim 1 further comprising collapsing the interior cavity.

15. The method of claim 14 further comprising injecting an adhesive or glue into the interior cavity to adhere the interior tissue to one another.

16. A system for closing a left atrial appendage, comprising:
a deployment catheter defining at least one lumen therethrough;
a barrier or membrane projecting distally from the deployment catheter and defining an open area therein, wherein the open area is in fluid communication with the at least one lumen;
a visualization element disposed within or along the barrier or membrane for visualizing tissue adjacent to the open area; and
a closure assembly deployable beyond the barrier or membrane within a cavity of the left atrial appendage.

17. The system of claim 16 further comprising a delivery catheter through which the deployment catheter is deliverable.

18. The system of claim 16 wherein the deployment catheter is steerable.

19. The system of claim 16 wherein the barrier or membrane is comprised of a compliant material.

20. The system of claim 16 wherein the barrier or membrane is adapted to be reconfigured from a low-profile delivery configuration to an expanded deployed configuration.

21. The system of claim 16 wherein the barrier or membrane is adapted to self-expand into the expanded deployed configuration.

22. The system of claim 16 wherein the barrier or membrane is conically shaped.

23. The system of claim 16 wherein the visualization element comprises at least one optical fiber, CCD imager, or CMOS imager.

24. The system of claim 16 wherein the closure assembly comprises an expandable mesh or scaffold configured to occlude an opening to the cavity of the left atrial appendage.

25. The system of claim 16 further comprising an ablation probe for ablating or scarring tissue around the opening and in contact with one another.

26. The system of claim 16 wherein the closure assembly comprises at least one pair of anchors connected to one another via a length of wire or suture for deployment into or through tissue surrounding an opening of the cavity.

27. The system of claim 26 further comprising a locking mechanism configured to slide uni-directionally along the wire or suture for approximating the pair of anchors towards one another.
